(19) United States
(12) Patent Application Publication
(10) Pub. No.: US 2025/0122561 A1
(43) Pub. Date: Apr. 17, 2025

(54) DEVICE FOR SEQUENCING A NUCLEOTIDE SEQUENCE EXHIBITING IN CREASE SENSITIVITY AND IM-PROVED RELIABILITY

(71) Applicant: COMMISSARIAT A L'ENERGIE ATOMIQUE ET AUX ENERGIES ALTERNATIVES, Paris (FR)

(72) Inventors: Thomas ALAVA, Grenoble Cedex 09 (FR); Jean-Pierre AIME, Bordeaux (FR); Sébastien HENTZ, Grenoble Cedex 09 (FR); Cédric HURTH, Sittingbourne (GB)

(73) Assignee: COMMISSARIAT A L'ENERGIE ATOMIQUE ET AUX ENERGIES ALTERNATIVES, Paris (FR)

(21) Appl. No.: 18/720,131

(22) PCT Filed: Dec. 14, 2022
(86) PCT No.: PCT/FR2022/052345

§ 371 Date: Dec. 31, 2024

(30) Foreign Application Priority Data
Dec. 16, 2021 (FR) FR2113618

(51) Int. Cl. C12Q 1/6874

(52) U.S. Cl. CPC C12Q 1/6874

(57) ABSTRACT

A device for sequencing at least one nucleotide strand including a support, at least one portion movable relative to the support, means for setting the movable portion in vibration at a given frequency, means for measuring the vibration frequency of the movable portion, and a recognition probe mechanically connecting the support and the movable portion, the recognition probe including at least one nucleotide sequence.

### TECHNICAL FIELD AND PRIOR ART

The present invention relates to a device for detecting at least one nucleotide sequence and to a detection system including a large number of such devices.

A sequencing device or sequencer is intended to determine the exact composition of a sequence of nucleic bases or nucleobases on an analysed DNA or RNA strand. This determination may be carried out by identifying the sequence by interaction with a probe formed by a strand. This interaction is reflected by an hybridisation reaction between the analysed strand and the probe strand. The analysed strand bonds more easily to a probe whose sequence corresponds exactly to the complementary sequence of the analysed strand, and less easily to a probe whose sequence is different from its complementary sequence.

The methods of the prior art lack sensitivity by generating a large number of false positives, i.e. cause pairing of two strands while their sequences are not complementary.

A method for reducing the number of false positives consists in reducing the length of the analysed strands, where this reduction results in increasing the duration and the cost of the analysis of the entire sequence.

Another method consists in increasing the number of sensors provided with the same recognition probe in order to carry out the same analysis, allowing statistically reducing the number of false positives. This method requires a considerable volume of samples

Moreover, there is great demand for providing DNA sequencing devices. In particular, rapid methods with reduced error rates.

### DISCLOSURE OF THE INVENTION

Consequently, the present invention aims to provide a detection device for sequencing at least one DNA or RNA fragment which is more sensitive and more reliable than the devices of the prior art.

More generally, the present invention relates to a device for detecting nucleic bases or nucleobases.

The aim set out hereinabove may be achieved by a detection or sequencing device including at least one fixed portion and one portion configured to be set in vibration relative to the fixed portion, an oligonucleotide probe mechanically connecting the fixed portion and the movable portion, means for setting the movable portion in vibration and means for measuring the vibration frequency of the movable portion before and after hybridisation of the analysed strand on the probe.

The level of hybridisation between the probe and the analysed strand makes the stiffness of the probe and analysed strand set vary, which has an influence on the mechanical resonance frequency of the vibrating portion. Thus, this hybridisation level is detected.

Depending on the variation of the mechanical resonance frequency, it is possible to determine whether the hybridisation is total, partial or zero and thus deduce the exact sequence of nucleobases of the analysed strand.

In other words, the mechanical stiffness of the set composed of an analysed strand and a nucleotide sequence is measured, this stiffness being characteristic of the level of pairing of the analysed strand and of the sequence, which allows deducing therefrom the structure of the strand in case of total pairing.

Advantageously, the movable portion is a resonating optomechanical ring.

The device may be integrated in a fluid channel in which a fluid including the analysed strands circulates so as to come into contact with the probe.

Preferably, a system including a large number of devices for detecting nucleic bases or nucleobases or for sequencing is made, allowing for example analysing a whole genome in one single measurement.

According to one aspect, the present invention relates to a device for detecting and/or sequencing at least one nucleotide strand including a support, at least one portion movable relative to the support, means for setting the movable portion in vibration at a given frequency, means for measuring the vibration frequency of the movable portion, a recognition probe mechanically connecting the support and the movable portion, the recognition probe including at least one nucleotide sequence.

Advantageously, the means for vibrating the movable portion are configured to set the movable portion in vibration at its resonance frequency.

According to a possible implementation, the means for measuring the vibration frequency consist of optomechanical means.

According to one embodiment, the means for measuring the vibration frequency include at least one waveguide optically coupled with the movable portion, a light source for injecting light into the waveguide and a unit for processing the light coming out of the waveguide.

Advantageously, the movable portion may be in the form of a disk, a ring, a hippodrome or an ellipse hanging from at least one foot.

According to a possible implementation, the device may include several identical probes connecting the movable portion to the support.

The support may include a first adapter precursor and a first adapter one end of which is fixed to the first adapter precursor and the other end includes a nucleotide sequence hybridised with one end of the recognition probe, and wherein the movable portion includes a second adapter precursor and a second adapter comprising a probe one end of which is fixed to the second adapter precursor and the other includes a nucleotide sequence hybridised with one end of the recognition probe.

The present invention also relates to a set including the device as defined before as well as a fluid channel ensuring contact of a liquid containing said analysed strand and the recognition probe.

The present invention also relates to a system including a plurality of sequencing devices as defined before.

Advantageously, the devices are distributed in groups, each group including recognition probes having the same nucleotide structure.

Advantageously, the recognition probes are selected so that a whole genome is analysed in one single measurement.

For this purpose, hundreds of thousands of sensors in parallel and/or several measurement cycles are typically provided for.

The present invention also relates to a method for detecting nucleic bases or nucleobases or for sequencing at least one nucleotide sequence including:
setting a liquid containing said sequence in contact with a resonating device including a recognition probe connecting a fixed portion and a movable portion of the resonating device,
setting the movable portion in vibration,
measuring the variation of the vibration frequency of the movable portion due to hybridisation between the recognition probe and the nucleotide sequence.

Advantageously, the movable portion is set in vibration at its resonance frequency.

Typically, the movable portion may be set in vibration with an amplitude in the range of 0.1 nm.

According to one embodiment, the movable portion is set in vibration at a frequency comprised between 100 MHz and 1 GHz.

### BRIEF DESCRIPTION OF THE DRAWINGS

The present invention will be better understood on the basis of the following description and the appended drawings wherein:

FIG. 1 is a side view of an embodiment of a device according to the invention,

FIG. 2 is a view of the device of FIG. 1 in a state wherein an analysed strand is hybridised on the probe,

FIG. 3B is a detail view of the device of FIG. 1,

FIG. 3A is a detail view of the device of FIG. 1 with no hooking probe,

FIG. 4 is a detail view of a variant of the device of FIG. 1,

FIG. 5 is a schematic illustration of the device of FIG. 1 integrated into a fluid channel,

FIG. 6 is an illustration of a device as implemented according to one embodiment and implementing optomechanical detection means,

FIG. 7 is a top view of a system comprising a network of detection devices,

FIG. 8A-8B, 9A-9B, 10A-10B is intended to illustrate the operation of an example of a device for detecting nucleic bases or nucleobases of a DNA or RNA strand and more particular for detecting the level of hybridisation between a recognition probe and this strand.

### DETAILED DISCLOSURE OF PARTICULAR EMBODIMENTS

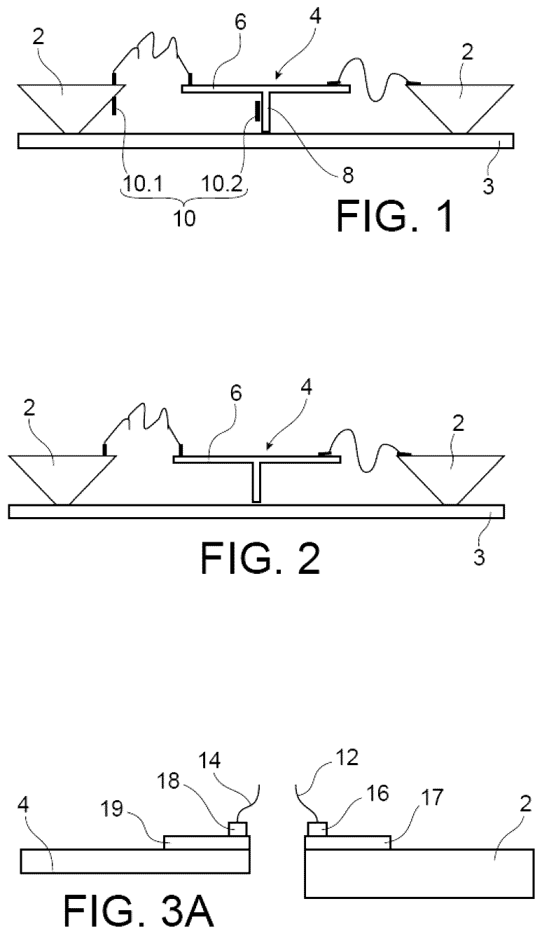

In FIG. 1, one could see an embodiment of a detection device. The invention applies to the determination of DNA fragments and RNA fragments, and can find application in genome sequencing.

DNA or RNA sequencing consists in determining the sequential order of the nucleotides for a given DNA fragment or a given RNA fragment, respectively.

In FIGS. 1, 2 and 3A and 3B, one could see an example of a device including at least one fixed portion 2, two in the illustrated example, for example secured to a support 3 and a portion 4, so-called movable portion, intended to be movable relative to the fixed portion 2 in the plane of the device. The fixed portion 2 is arranged proximate to the movable portion 4. For example, the distance between the edge of the fixed portion 2 and the edge opposite the movable portion 4 is comprised between 100 and 200 nm. This distance is selected so as to avoid any contact between the movable portion and the fixed portion in normal conditions of oscillation of the movable portion.

The plane of the device or of the sensor is the plane parallel to the support 3, generally formed by a substrate implemented in microelectromechanics.

In the illustrated example, the movable portion includes a platform 6 hanging from a foot 8. The dimensions of the foot and of the platform 6 are such that the platform 6 can oscillate substantially in the plane.

The device is a resonating device and includes excitation means 10 for setting the movable portion 4 in vibration in the plane. In the example schematically illustrated in FIG. 1, the excitation means are of the electrostatic type and include an electrode 10.1 on the fixed portion and an electrode 10.2 in the movable portion formed by the foot directly. The application of a potential difference between the electrodes generates electrostatic forces between the foot and the fixed portion setting the platform in vibration. The implementation of excitation means at the foot allows limiting bulk.

Alternatively, it is also possible to use optical excitation means. In particular, radiation or thermo-optical forces are used. In both cases, this consists in using a laser with a wavelength in an optical peak, but whose power is modulated at the frequency of interest, in this case that one of mechanical resonance of the device. This laser may be the same as that one used for optical readout, or another laser, for example superposed with the first one.

The device also includes means (not shown) for measuring the vibration frequency of the movable portion. The movable portion then forms a mechanical resonator. For example, these means include one or more capacitive transducers, piezo-resistive transducers, optomechanical transducers, acoustic wave transducers, of the bulk acoustic wave type BAW (“Bulk Acoustic Wave” in Anglo-Saxon terminology) or of the surface acoustic wave type SAW (“Surface Acoustic Wave” in Anglo-Saxon terminology).

The movable portion 4 may be a beam embedded by one end and set in vibration by electrostatic means and whose frequency is measured by piezoresistive gauges.

According to another example, the movable portion 4 may be a plate vibrating in a Lame or breathing mode.

An example of optomechanical detection will be described in details.

Preferably, the movable portion is excited at a resonance frequency enabling a maximum amplification of the input signal and a maximum sensitivity of the response of the device to a disturbance (modification of the environment).

Preferably, the resonator is selected so as to have reduced viscous damping. Indeed, the device is intended to be arranged in a fluid channel n which a liquid containing the analysed strand(s) flows.

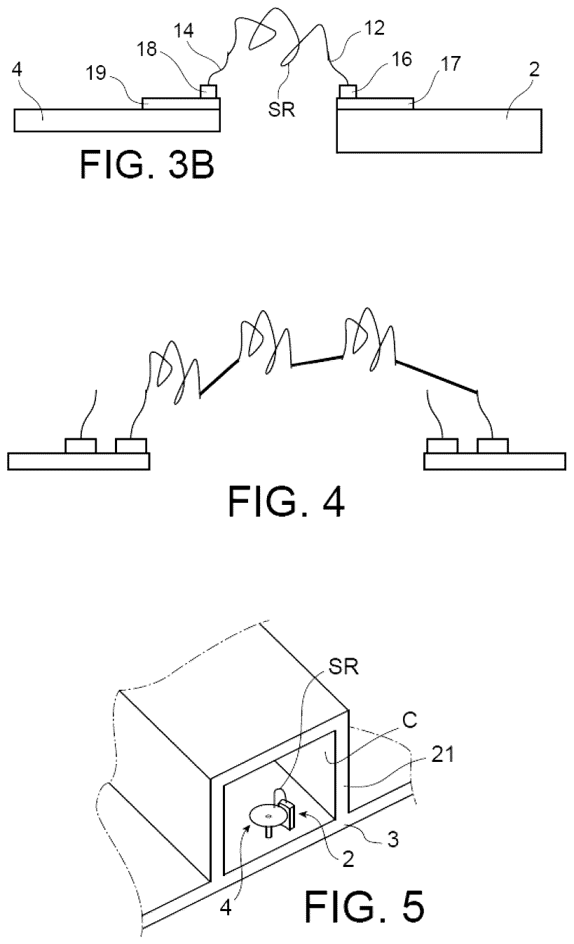

In FIG. 3B, one could see an enlarged view of the device of FIG. 1. FIG. 3A shows the device before a recognition probe SR is fixed.

The device includes a recognition probe SR intended to hybridise with the analysed strand. For example, the recognition probe is an oligonucleotide probe. For example, the probe may include between one hundred and several thousands nucleotides.

The device also includes nucleotide sequences 12, 14 one fixed by one end to the fixed portion 2 and the other one fixed by one end to the movable portion 4. The two free ends of the nucleotide sequences 12, 14 are intended to hook at the ends of the recognition probe SR by hybridisation and enable the recognition probe SR to mechanically connect the fixed portion 2 and the movable portion 4.

The sequences 12, 14 are so-called “adapters” and include the sequences complementary to the ends of the recognition probe, which enables hybridisation of the two ends of the recognition probe on the adapters. Once hooked to the adapters 12, 14, the recognition probe SR includes a series of free nucleotides, so-called “useful area” and intended to cooperate with the analysed strand by hybridisation.

The fixed portion includes, over its upper face, a layer 16 enabling hooking of the adapter 12 and the upper face of the movable portion includes a layer 18 enabling hooking of the adapter 14. The layers 16, 18 are so-called “adapter precursors” and include, for example, silane or a thin metal layer. A DNA-thiol adapter on a hooking layer made of gold may be provided. One or more thiol(s), polyethylene glycol PEF, epoxide may also be used for the adapter precursor. Moreover, there is string affinity between biotine and streptavidine, thus, one of these elements may correspond to an adapter precursor whereas the other one may be provided at one end of the adapter 12, 14 thereby allowing “attaching” the adapter on the fixed or movable portion.

Preferably, prior to deposition of the adapter precursors, the upper face of the fixed portion and of the movable portion have different functionalisations in order to be selective with regards to the adapter precursors. For example, the functionalisation is obtained by forming a film 17, over the upper face of the fixed portion, and film 19 made of a different material over the upper face of the movable portion. For example, the films 17, 19 may be made of a polymer material or of a metal material such as gold. A particular embodiment provides for:
a film 17 made of SiO₂, and a layer 16 made of PLL-g-PEG or silane
a film 19 made of gold with a thiol-based layer 18.

Typically, the films 17, 19 have a thickness in the range of 100 nm.

Preferably, the functionalised areas are located at the most mechanically sensitive locations, i.e. which have the greater displacement, for example the edge of the movable portion and/or opposite the fixed portion.

Hence, the adapters are selected according to the recognition probe to be arranged between the fixed portion and the movable portion and therefore according to the analysed strand.

Advantageously, the device includes several fixed portions and several recognition probes connecting the fixed portions to the movable portion, the probes including the same nucleotide sequences. The fixed portions may be made in one-piece and for example formed by a ring around the movable portion. By increasing the number of recognition probes, the probability of hooking of the analysed strand(s) is increased, which increases the sensitivity of the device.

In FIG. 5, one could see a schematic illustration of a set including the device of FIGS. 1 and 2 integrated in a fluid channel C, for example formed by cooperation between a cover 21 and the support 3 of the device. The circulation of the liquid containing the strands to be analysed is done in open circuit or in continuous circuit. In open circuit, the channel is connected to a tank containing the liquid and the strands to be analysed and to an area for collecting the liquid after circulation in the channel. A circulation pump may be provided.

In closed circuit, the liquid is, for example, injected into the channel by a syringe and a pump ensures recirculation of the liquid in the channel. The closed-loop operation allows recycling the products and carrying out several measurements. It is possible to provide for resetting the sensors by implementing a thermal melting of the paired DNA strands. It is also possible to provide for accumulating measurement data without performing any intermediate heating between the consecutive measurement cycles by making the fluid circulate several times on the device(s).

The operation of this device will now be described.

The movable portion 4 connected to the fixed portion 2 by the “adapter—recognition probe—adapter” set is set in vibration by the excitation means. The amplitude of the vibration in the range of an interatom distance, about 0.1 nm (1 Angström). The “adapter—recognition probe—adapter” set has a determined stiffness and dissipates a determined amount of energy. The movable portion oscillates in the plane at a given frequency, typically between 100 kHz and 1 GHZ, preferably between 1 MHz and one or several tens MHz which results from the excitation means, the stiffness of the “adapter—recognition probe—adapter” set and its dissipation.

A liquid containing the analysed DNA or RNA strand circulates on the sequencer device. The latter comes in contact with at least the portion of the recognition probe SR that is not hybridised with the adapters. The analysed strand hybridises more or less completely with the hooking probe (FIG. 2). The higher the hybridisation level, the more the stiffness of the “adapter—recognition probe—adapter” set and of the analysed strand increases and the more the probe-strand set couples the fixed portion to the movable portion which results in modifying the oscillation frequency of the movable portion. The measurement means measure this vibration frequency which has varied relative to its vibration frequency before hybridisation of the analysed strand. The frequency may vary from one to several hundred Hertz.

Indeed, when the analysed strand hybridises with the recognition probe, the two strands are contracted and the set thus formed has a determined and known longitudinal stiffness, higher than that of the recognition probe alone. The great measurement sensitivity, within a margin of one single nucleotide, SNP (standing for “single-nucleotide polymorphism”, i.e. a polymorphism of one single nucleotide), may be proposed in the implementation of a strand displacement type kinetic competition. Hence, the device has a great reliability, a great sensitivity and a high detection speed.

The measurement of the dissipated energy is rich in information on the hybridisation process.

In the case where no hybridisation occurs between the analysed strand and the hooking probe, the vibration frequency of the movable portion does not vary or does not vary significantly, in particular a vibration by less than 0.001% which corresponds for example to a vibration of 300 Hz for an initial resonance frequency of 30 MHz.

The great sensitivity of the detection means allows detecting the level of hybridisation between the strand and the recognition probe. Nevertheless, the fact that the device allows detecting and measuring the intermediate hybridisation states (partial hybridisation), allows obtaining selective information on the different sequences.

This device allows for a greater detection reliability.

Furthermore, the sequencer device enables analysis of long sequences of nucleotides, which allows reducing the analysis time. For example, about 5*10⁶ pairs of bases are provided for to analyse one bacterium.

Advantageously, mechanically loading the hybridised strand allows verifying the hybridisation. If the strand is partially hybridised, the hybridisation will be less resistant to an axial mechanical load. The axial load then allows substantially reducing the risk of false positives.

The method for detecting the hybridisation level could then take place according to the following steps:
Setting the resonator in vibration at a given frequency, preferably its mechanical resonance frequency,
Circulating a solution containing the strand to be analysed on the device,
Measuring the vibration frequency of the resonator,
Detecting any perfect hybridisation, partial hybridisation or no hybridisation.
If there is a perfect hybridisation, the sequence of the analysed strand is then known.

Preferably, the liquid circulates during hybridisation.

Preferably, the circulation speed of the liquid is limited, for example to a flow rate in the range of one or several ml/min. The hybridisation may last about several minutes, for example 5 minutes.

It is possible to tune the flow speed, the repetition of the measurements with the cycles, perform stages. The circulation of the liquid is stopped and the measurement is performed by accumulation of the data with a probability of hooking controlled by the diffusion of the targets [Note: an addition option which enables different measurement types without disturbing the flow].

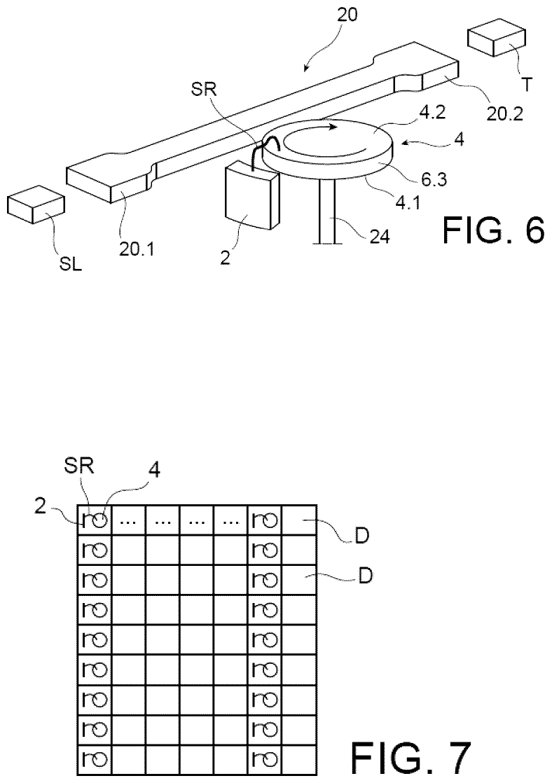

In FIG. 6, one could see an embodiment wherein the detection means consist of optomechanical means.

The device includes an optical resonator 4, at least one waveguide 20 optically coupled with the resonator, the waveguide being supported by the substrate, a light source SL and means T for processing the light wave coming out of the waveguide. The support, the waveguide, the optomechanical resonator forms a sensor structure.

The waveguide 20 includes an input end 20.1 if a light wave connected to a light source via a coupling network 22.1 not shown in the figure, and an output end 20.2 connected to means for processing the light wave coming out of the waveguide via a coupling network 20.2.

The resonator 4 is arranged proximate to a sidewall of the waveguide 20 so as to be optically coupled to the latter. The waveguide 20 is in the evanescent field of the resonator, so that the light wave originating from the source SL is injected in the optical resonator and the light wave having circulated in the resonator is collected by the waveguide. The wave circulating in the resonator is symbolised by an arrow.

For example, the width of the space between the sidewall of the waveguide and the lateral edge of the resonator is comprised between 10 nm and 50 nm.

In the illustrated example, the optomechanical resonator 4 is in the form of a disk hanging from a foot 24 fastened to a face of the disk opposite the substrate. The disk extends in a plane of the sensor. The resonator includes two end faces 4.1, 4.2 substantially parallel to the plane of the sensor and one lateral face 6.3 (FIG. 6).

Preferably, the foot has a smaller diameter compared to the dimensions of the disk in the plane of the sensor, more particularly a smaller diameter compared to the diameter of the disk, preferably the foot has a diameter at least 10 times as small as the diameter of the disk.

More generally, the diameter of the foot is ten times as small as the smallest dimension of the resonator in the plane of the sensor, thus the foot barely disturbs, or not all, the radial vibration of the resonator.

Alternatively, the resonator is suspended by springs in the plane or by nanometric beams extending radially and compressed and tensioned by the vibration of the disk. The springs or the beams are then sized so as to have a lower axial stiffness than that of the resonator.

Any other resonator shape may be suitable, for example the resonator may have a ring-like, ellipse-like or hippodrome-like (“racetrack” in Anglo-Saxon terminology) shape, when seen in top view. In the case of a ring-like, ellipse-like or hippodrome-like shaped resonator, the latter hangs from the substrate for example by means of a foot located at the centre of the resonator, and beams extending between the internal edge of the resonator and the foot.

The resonator may be made of any material capable of confining an electromagnetic wave, such as GaAS, Ge or Gi. The latter is particularly interesting for manufacture according to microelectronics techniques offering a high level of integration on a substrate.

For example, the functionalisation layer is formed over the face 4.2 of the resonator opposite to that one 4.1 opposite the substrate.

For example, the excitation means consist of electrostatic means (not shown) or optical means.

The operation of the sensor will now be described.

Preferably, the wavelength of the light wave to be injected in the resonator is selected close to the optical resonance of the resonator, i.e. at the edge of the optical resonance peak. The light resonating inside the optical resonator is then very sensitive to the mechanical deformation of the mechanical resonator, in particular when the optical and mechanical resonator are combined.

The light wave at the selected wavelength is injected in the waveguide by the light source, by optical coupling, the light wave is injected in the optomechanical resonator 4.

When an analysed strand hybridises on the recognition probe, depending on the hybridisation level, the stiffness of the connection between the resonator and the fixed portion is modified and the dissipated energy too, which has an effect on the vibration frequency of the resonator, which generally increases.

Indeed, the mechanical frequency ƒ=\sqrt{stiffness/mass}

The mass of the resonator almost does not vary upon hybridisation, consequently, when the stiffness of the resonator increases, its frequency increases.

The measurement of the variation of the vibration frequency allows determining whether the hybridisation was total or not, and if the hybridisation was total, determining the identity of the analysed strand. Indeed, the optomechanical resonator is sensitive enough to distinguish between a perfect or almost perfect pairing.

Alternatively, the measurement of the variation of the frequency may be combined with a measurement of the variations of the optical properties of the resonator thereby allowing acquiring complementary information.

For example, the optomechanical resonator has a vibration frequency between 100 MHz and 1 GHz with amplitude oscillations in the range of a few picometres.

Working at high frequencies allows increasing the detection sensitivity and the accuracy of the gathered data in particular by a time sampling level never reached hitherto.

The optomechanical resonator as described hereinabove further offer the advantage of allowing for very good performances in a liquid medium.

Preferably, a system comprising a large number of detection devices D (FIG. 7) is made. For example, it is possible to provide between 9 and about 10,000 resonators. An even greater number of resonators may be provided, the limit could be defined according to the manufacturing cost.

Each device or each group of devices may be provided with different recognition probes. Each detector outputs its own signal which is processed by a processing unit. Only part of the devices is schematised.

For example, the system includes a fluid channel that is common to all devices or several parallel fluid channels, each supplying one column of devices. For example, the channel or the channels is/are formed in a cover (not shown).

The implementation of optomechanical means facilitates such an integration thanks to multiplexing techniques well developed in the optics field.

Furthermore, the implementation of microelectronics manufacturing techniques enables the integration of a very large number of devices.

The recognition probe equipping each device is known since it depends on the adapters fixed on the fixed portion and the movable portion which are themselves determined by the functionalisation of each of the portions. Hence, the analysed strand detectable by each device is known.

By spatially locating the probes and therefore the responses given by each of the devices, it is possible to carry out redundant measurements that could be exploited, and to obtain low error rates.

The above-described method applies to a sequencing system, however, such a system allows detecting several different strands. The liquid then contains genome fragments, for example a genome of several thousands bases. The latter is injected into the channel or the channels of the system and after a potential step of hybridisation of the strands on the appropriate recognition probes, the vibration frequencies of the different resonators are measured.

The frequencies may be measured continuously, allowing monitoring the hybridisation process.

The detection device and the detection system can be reused; after a sequencing cycle, they may be rinsed in order to remove the strands hybridised on the recognition probes SR.

One example of a rinsing method uses as a rinsing solution a DNA-free buffer solution (“buffer” according to the Anglo-Saxon terminology), for example of the tris-acetate, tris-EDTA, PBS (“Phosphate Buffered Saline”), HEPES (N-(2-Hydroxyethyl)piperazine-N′-(2-ethanesulphonic acid) type with the addition of a surfactant, typically in a small amount, i.e. in the range of 0.1% or lower. The surfactant may consist of SDS (“Sodium dodecyl sulphate”) or of TWEEN (Polyethylene glycol sorbitan monolaurate). The rinsing solution is circulated to unhook the DNA strands hybridised on the recognition probe. Preferably, the circulation should not be done in a closed circuit. This example is given only for illustrative purposes, a person skilled in the art should know how to adapt the components of the rinsing solution according to the nature of the DNA strands and other reagents used.

It could also be considered to replace the recognition probes, these may be removed and replaced by other probes in order to modify the application of the device or of the system.

By raising the temperature within a range comprised for example between 50 and 80° C., it is possible to obtain unhooking of the probes from the hooks and the hooks become free to place new probes.

It should be noted that the resonators could be set in vibration at different frequencies, for example they may have different mechanical resonance frequencies.

For example, by using recognition probes with a 50-micrometre linear dimension, for a sequencing system including groups of 200 devices provided with the same recognition probe comprising 150 pairs of nucleotides. The sequencing system comprises 4,104 optomechanical detectors/cm², i.e. about 200 groups of 200 devices having the same recognition probe and therefore capable of detecting 200 different sequence domains. This system then allows analysing a viral whole genome of 30 kb, i.e. 30,000 bases or nucleotides, in one single measurement series.

In another example, each device has a side measuring 40 μm, this allows making sequencing systems having a density of devices of 62.5×10³ cm².

As already mentioned hereinabove, the detection device and the detection system may advantageously be made at least in part by microelectronics techniques.

The movable portion(s) and the fixed portion(s) and possibly the waveguides are made by deposition of layers over a substrate and lithography and etching. The electrical tracks are also made by deposition and etching.

For example, the polymer layers as well as the adapter precursors are deposited and localised by lithography and etching.

For example, the cover is made of glass and is structured so as to form the fluid channel(s) with the substrate.

The cover is affixed on the substrate and is, for example, bonded to the latter. During a next step, a liquid containing the adapters is injected into the channel(s). The liquid is a physiological liquid. The adapters are then fixed on the adapter precursors. During the hooking phase, the liquid is at rest or circulating generating a shear force on the fixed and movable portions.

Typically, if no circulation is implemented, a sedimentation and a poor reading, with the risk of false positives might happen. Nonetheless, if an excessively rapid liquid circulation is implemented, a poor hooking resulting in the risk of false negatives might happen. A person skilled in the art should know how to adapt the liquid circulation speed. For example, it is possible to provide for a speed between 1 ml/min and several mL/min.

During a next step, the physiological liquid containing the recognition probes is injected in the channel(s). The probes are fixed by one end to an adapter on the fixed portion and by another end on the movable portion. During the phase of hooking of the probes, the liquid is at rest or circulating generating a shear force on the fixed and movable portions.

It should be noted that the relative arrangements of two neighbouring devices are such that there is no risk of a probe being fixed on a movable portion of one device and on a fixed portion of a fixed portion of another device.

The detection device and the detection system have the advantage of using well-known materials and techniques. They have a good robustness.

Furthermore, the combination of a large number of sequencing devices enables numerous applications, such as the parallel treatment and profiling of coding sequences, the selection and localisation of genes, the parallel treatment of multiple mutations, the selection and the localisation of viral variants.

A device for detecting nucleic bases or nucleobases of a DNA or RNA strand as described before allows detecting a level of hybridisation between a recognition probe and a given sequence of nucleotides. The more the recognition probe and the sequence are complementary, the more significant the hybridisation level will be.

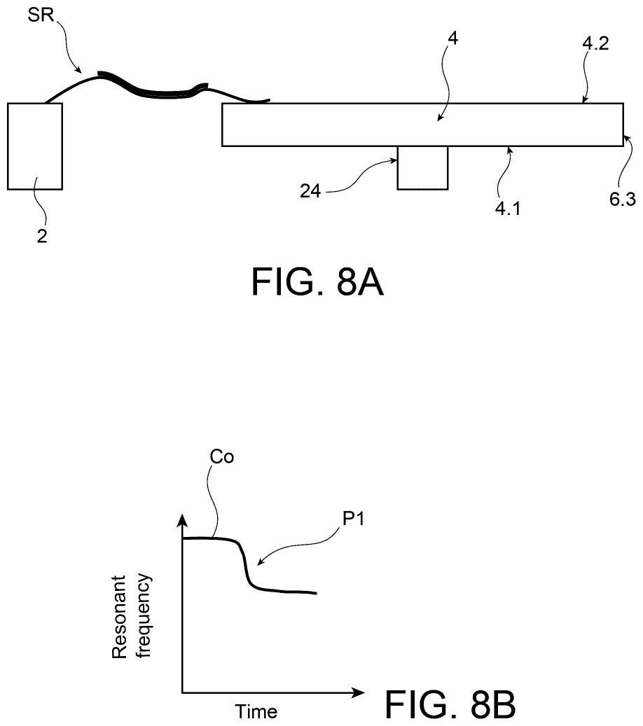

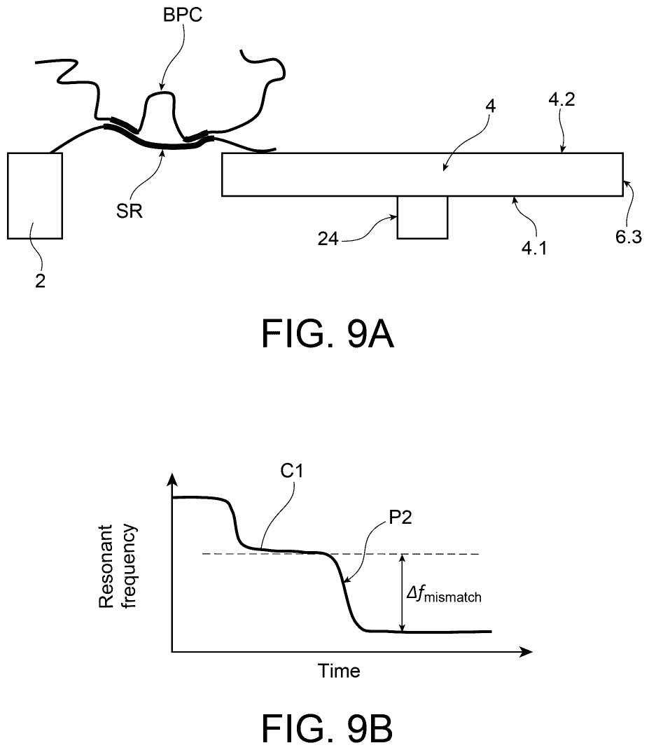

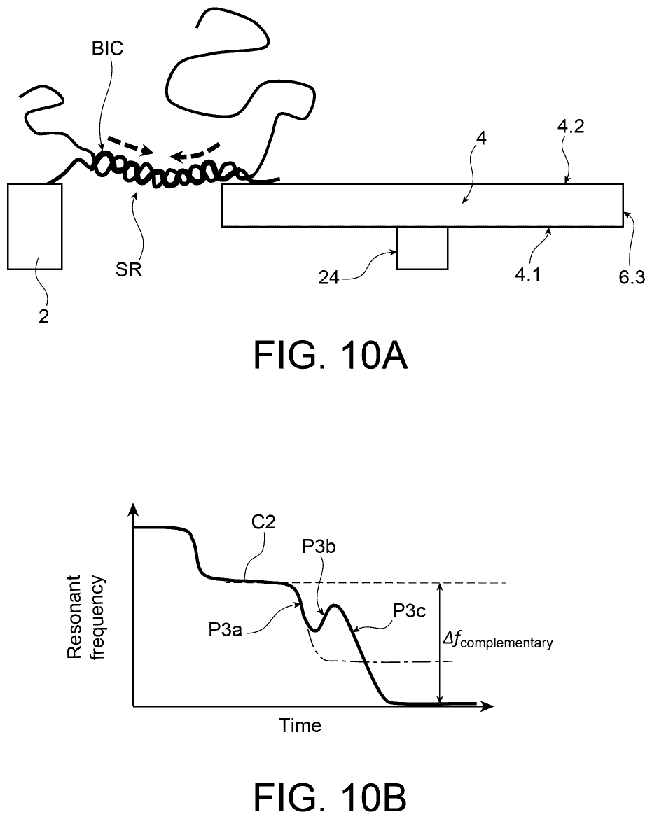

The device illustrated in FIG. 8A replicates an example of arrangement previously described in connection with FIG. 6. The genetic probe SR is affixed on the one hand to a fixed portion 2 and on the other hand to a movable portion 4, for example in the form of a disk, and in particular a surface 4.2 of this disk. The addition of the probe SR on the resonating sensor 4 causes an increase in the vibrating mass and, as illustrated in FIG. 8B (schematically showing a curve of the evolution Co of the resonance frequency of the disk 4 as a function of time) a decrease in the resonance frequency (portion P1 in the curve Co). Such an arrangement could induce a lesser downward offset of the resonance frequency compared to a conventional arrangement where the genetic probe SR would be entirely attached on the movable portion 4.

Indeed, with an arrangement as proposed where the probe SR is affixed on the one hand to the disk 4 and on the other hand to the fixed portion 2, the mass of the probe SR is less coupled with the vibratory movement of the disk 4 on its portion close to the fixed portion 2.

FIGS. 9A-9B are intended to illustrate the case when a so-called “partially complementary” DNA strand BPC is bonded to the probe SR because this strand BPC includes one or more base(s) in common with a target region of the probe SR.

The addition of the mass of the partially complementary strand BPC to the vibratory system consisting of the disk 4 and the probe SR results in a downward offset of the resonance frequency (portion P2 of the resonance frequency curve, the offset being equal to Δfmismatch) related to a mere mass effect. There is no more force exerted by the probe SR on the movable portion 4 other than the mass in this case, subsequent to hooking of this partially complementary strand BPC.

In the example illustrated in FIGS. 10A-10B, a perfectly complementary DNA strand BIC hybridises to the probe SR because it includes the sequence exactly complementary to the sequence targeted by the probe SR. The first effect (portion P3a of the resonance frequency curve C2) consists in a mass increase reflected by a decrease in the resonance frequency related to attachment of the complementary strand with the probe SR on a few bases, thereby coupling the mass of the strand BIC to that of the probe SR and the movable portion 4. The more hooking of the complementary nucleobases progresses, the more a double-stranded double DNA helix tends to form. The formation of a double helix, possibly perfect, related to the complementarity, possibly perfect, of the two sequences, will generate a tensile force exerted by this double helix on the movable portion 4 through hooking of the probe SR on the surface 4.2. This additional tensioning force confers a greater stiffness on the system by the recognised strand BIC—the probe SR—the movable portion 4 which is reflected by a re-increase in the resonance frequency in this case (the portion P3b). Finally, as the double helix between the recognised strand and the probe SR is made, the mechanical coupling between the mass of the two strands, the recognised strand and that one of the probe SR respectively, increases and the vibratory movement of the movable portion 4 decreases which results, at the end of the experiment, in a decrease in the resonance frequency (final offset of the resonance frequency Δfcomplementary) with a greater intensity than is the case for the offset Δfmismatch of the previous example with a partially complementary strand. Thus, the resonance frequency offset reflects the level of hybridisation.

It is possible to carry out a calibration of the system by measuring the curve C2 with the strand BIC. In this manner, during the next measurements, it is possible to immediately find out whether an entirely compatible strand BIC or a partially compatible strand BPC is detected.

A detection device and a detection system as described before apply to sequencing of at least one nucleotide strand.

1. A device for detecting at least one nucleotide strand, the device including:
a support,
at least one portion movable relative to the support,
means for setting the movable portion in vibration at a given frequency,
means for measuring the vibration frequency of the movable portion, and
a recognition probe mechanically connecting a fixed portion secured to the support and the movable portion, the recognition probe including at least one nucleotide sequence.

2. The device according to claim 1, wherein the means for vibrating the movable portion are configured to set the movable portion in vibration at a resonance frequency of the movable portion.

3. The device according to claim 1, wherein the means for measuring the vibration frequency consist of optomechanical means.

4. The device according to claim 3, wherein the means for measuring the vibration frequency include at least one waveguide optically coupled with the movable portion, a light source for injecting light into the waveguide and a unit for processing the light coming out of the waveguide.

5. The device according to claim 4, wherein the movable portion is a disk, a ring, a hippodrome, or an ellipse hanging from at least one foot.

6. The device according to claim 1, including several identical probes connecting the movable portion to the support.

7. The device according to claim 1, wherein the fixed portion includes a first adapter precursor and a first adapter, one end of which is fixed to the first adapter precursor and the other end includes a nucleotide sequence hybridised with one end of the recognition probe, and
wherein the movable portion includes a second adapter precursor and a second adapter comprising a probe, one end of which is fixed to the second adapter precursor and the other includes a nucleotide sequence hybridised with one end of the recognition probe.

8. The device according to claim 1, wherein the means for setting the movable portion in vibration at a given frequency are of the electrostatic type, and include one electrode on the fixed portion and one electrode on the movable portion.

9. A set, including:
the device according to claim 1; and
a fluid channel ensuring contact of a liquid containing said analysed strand and the recognition probe.

10. A system including a plurality of devices according to claim 1.

11. The system according to claim 10, wherein the devices are distributed in groups, each group including recognition probes having the same nucleotide structure.

12. The system according to claim 11, wherein the recognition probes are selected so that a whole genome is analysed in one single measurement.

13. A detection method for sequencing at least one nucleotide sequence, the method including:
setting a liquid containing said sequence in contact with a resonating device including a recognition probe connecting a fixed portion and a movable portion of the resonating device,
setting the movable portion in vibration, and
measuring a variation of a vibration frequency of the movable portion due to hybridisation between the recognition probe and the nucleotide sequence.

14. The method according claim 13, wherein the movable portion is set in vibration at a resonance frequency of the movable portion.

15. The method according to claim 13, wherein the movable portion is set in vibration with an amplitude in the range of 0.1 nm and/or wherein the movable portion is set in vibration at a frequency between 100 MHz and 1 GHz.
